(19) United States
(12) Patent Application Publication
(10) Pub. No.: US 2002/0082515 A1
(43) Pub. Date: Jun. 27, 2002

(54) Thermography catheter

(76) Inventors: Thomas H. Campbell, Brentwood, CA (US); William L. Sweet, Mountain View, CA (US); David A. Rahdert, San Francisco, CA (US)

Correspondence Address: Steve D. Beyer, Beyer Weaver & Thomas, LLP, P.O. Box 778, Berkeley, CA, 94704-0778, US

(21) Appl. No.: 09/848,779

(22) Filed: May 3, 2001

Related U.S. Application Data
(63) Continuation-in-part of application No. 09/346,072, filed on Jul. 1, 1999, now Pat. No. 6,245,026.
(60) Provisional application No. 60/201,608, filed on May 3, 2000.

(51) Int. Cl. A61B 5/00

(52) U.S. Cl. 600/549

(57) ABSTRACT

Interventional tools are described that are suitable for measuring the temperature of or temperature variations in a vessel wall in the body of a patient and thereafter treating vulnerable plaque that is identified during the thermal mapping. The described interventional tools all include one or more thermal sensors that are suitable for detecting an indication of the temperature of or temperature variations in walls of a vessel the tool is inserted into. These sensors may be used to facilitate the detection of vulnerable plaque within the vessel. In one aspect, the interventional tool includes a stent delivery device that is suitable for delivering a stent to a selected segment of a vessel the interventional tool is inserted into. In an alternative aspect the interventional tool includes a deployment lumen. The deployment lumen is sized suitably for receiving a stent delivery catheter therethrough. A distal port that opens from the deployment lumen permits the distal portion of the stent delivery catheter to pass therethrough and to exit the elongated member to permit deployment of a stent.

In another quite different arrangement, a heating element is provided. The heating element is arranged to heat a segment of a vessel that is identified as containing vulnerable plaque. Preferably, the heating element heats the vessel walls to a temperature sufficient to induce apoptosis in inflammatory cells associated with the vulnerable plaque.

This application claims the benefit of U.S. Provisional Application No. 60/201,608 filed on May 3, 2000, the disclosure of which is incorporated herein by reference.”

### FIELD OF THE INVENTION

The present invention relates generally to medical devices suitable for thermally mapping body vessel segments to locate hot spots (areas with elevated temperatures associated with high metabolic activity and/or inflammation) within the vessel. More particularly thermography catheters that include treatment capabilities including stent delivery and/or thermal heating are described.

### BACKGROUND OF THE INVENTION

Cardiovascular disease is one of the leading causes of death worldwide. For example, some recent studies have suggested that plaque rupture may trigger 60 to 70% of fatal myocardial infarctions. In a further 25 to 30% of fatal infarctions, plaque erosion or ulceration is the trigger. Vulnerable plaques are often undetectable using conventional techniques such as angiography. Indeed, the majority of vulnerable plaques that lead to infarction occur in coronary arteries that appeared normal or only mildly stenotic on angiograms performed prior to the infarction.

Studies into the composition of vulnerable plaque suggest that the presence of inflammatory cells (and particularly a large lipid core with associated inflammatory cells) is the most powerful predictor of ulceration and/or imminent plaque rupture. For example, in plaque erosion, the endothelium beneath the thrombus is replaced by or interspersed with inflammatory cells. Recent literature has suggested that the presence of inflammatory cells within vulnerable plaque and thus the vulnerable plaque itself, might be identifiable by detecting heat associated with the metabolic activity of these inflammatory cells. Specifically, it is generally known that activated inflammatory cells have a heat signature that is slightly above that of connective tissue cells. Accordingly, it is believed that one way to detect whether specific plaque is vulnerable to rupture and/or ulceration is to measure the temperature of the plaque walls of arteries in the region of the plaque.

Once vulnerable plaque is identified, the expectation is that in many cases it may be treated. Since currently there are not satisfactory devices for identifying and locating vulnerable plaque, current treatments tend to be general in nature. For example, low cholesterol diets are often recommended to lower serum cholesterol (i.e. cholesterol in the blood). Other approaches utilize systemic anti-inflammatory drugs such as aspirin and non-steroidal drugs to reduce inflammation and thrombosis. However, it is believed that if vulnerable plaque can be reliably detected, localized treatments may be developed to specifically address the problems.

Recently there have been several efforts to develop thermography catheters that are capable of thermally mapping vascular vessels to identify thermal hot spots that are indicative of vulnerable plaque. By way of example, commonly assigned U.S. patent application Ser. No. 09/346,072, filed Jul. 1, 1999(which is incorporated herein by reference) describes a number of thermography devices and combined thermography and drug delivery and/or sampling catheters. Other thermography catheters are described in U.S. Pat. No. 5,871,449 (to Brown), U.S. Pat. No. 5,935,075 (Cassells et al.) and U.S. Pat. No. 5,924,997 (Campbell), each of which are incorporated herein by reference.

Recent experiments have shown that thermography is indeed capable of thermally mapping a vessel to the degree necessary to identify vulnerable plaque. However for thermography to become popular, it is going to be critical to develop localized treatments that can be administered when vulnerable plaque is identified.

In view of the foregoing, improved catheters that combine the identification, location and mapping of inflamed plaque and/or other hot spots within arteries and/or other vessels with various treatment capabilities would be desirable.

### SUMMARY OF THE INVENTION

To achieve the foregoing and other objects of the invention, interventional tools are described that are suitable for measuring the temperature of or temperature variations in a vessel wall in the body of a patient and thereafter treating vulnerable plaque that is identified during the thermal mapping. The described interventional tools all include one or more thermal sensors that are suitable for detecting an indication of the temperature of or temperature variations in walls of a vessel the tool is inserted into. These sensors may be used to facilitate the detection of vulnerable plaque within the vessel.

In one aspect, the interventional tool includes a stent delivery device that is suitable for delivering a stent to a selected segment of a vessel the interventional tool is inserted into. In an alternative aspect the interventional tool includes a deployment lumen. The deployment lumen is sized suitably for receiving a stent delivery catheter therethrough. A distal port that opens from the deployment lumen permits the distal portion of the stent delivery catheter to pass therethrough and to exit the elongated member to permit deployment of a stent.

In another quite different arrangement, a heating element is provided. The heating element is arranged to heat a segment of a vessel that is identified as containing vulnerable plaque. Preferably, the heating element heats the vessel walls to a temperature sufficient to induce apoptosis in inflammatory cells associated with the vulnerable plaque. The heating element may take a variety of forms. By way of example, the heating element may be an antenna suitable for delivering electromagnetic energy (such as microwave energy) to facilitate heating. Alternatively a resistive heater or other suitable heating element may be used.

In a system aspect of the invention, a display device may be electrically coupled to the interventional tool and be arranged to receive the signals from the thermal sensors. The display device is preferably arranged to display a thermal map of a longitudinal section of the vessel that shows temperature variations along the vessel to facilitate identifying a region of vulnerable plaque.

### BRIEF DESCRIPTION OF THE DRAWINGS

The invention, together with further objects and advantages thereof, may best be understood by reference to the following description taken in conjunction with the accompanying drawings in which:

FIG. 1 illustrates a combination thermal mapping and stent delivery catheter in accordance with one embodiment of a first aspect of the present disclosure.

FIG. 2 illustrates a combination thermal mapping and thermal heating catheter in accordance with an embodiment of a second aspect of the present disclosure.

FIG. 3 diagrammatically illustrates the catheter of FIG. 1 with the thermal sensor carrying balloon inflated to facilitate thermal mapping of a section of a section of a vascular vessel to identify vulnerable plaque 61.

FIG. 4 diagrammatically illustrates the distal end of the catheter of FIG. 1 with the stent delivery device 70 deployed within the vessel and expanded to deliver a stent 71.

FIG. 5 diagrammatically illustrates the catheter of FIG. 2 with the thermal sensor carrying balloon inflated to facilitate heating of the artery walls in the region of identified vulnerable plaque 61.

FIG. 6 illustrates a combination thermal mapping and stent delivery catheter in accordance with a second embodiment of the first aspect of the present disclosure.

FIG. 7 illustrates the distal end of the catheter of FIG. 6 with the stent delivery mechanism deployed within the vessel and expanded to deliver a stent 71.

FIG. 8 is a diagrammatic representation of a monitor having a screen displaying a thermal map taken using one of the described thermal mapping catheters.

FIG. 9 is a side view of a proximal hub assembly suitable for use with some of the described catheters.

### DETAILED DESCRIPTION OF THE INVENTION

Several presently preferred interventional devices suitable for detecting vulnerable plaque and then treating the affected region will be described below making reference to the accompanying drawings. Generally, the described interventional devices include thermal mapping catheters and are intended to permit the diagnosis of body vessel regions that have relatively higher heat production compared with surrounding tissues and/or the temperature of adjacent luminal fluid (e.g. blood passing through an vessel (e.g. artery) being mapped). These thermal mapping capabilities are combined with other therapeutic capabilities to provide integrated tools for diagnosis and/or treatment of specific conditions. For the purpose of illustration, the inventions will be described in the context of catheters and methods suitable for thermally mapping vulnerable plaque in vascular vessels such as coronary arteries.

Generally, there are a number of considerations that must be addressed when designing a thermal mapping catheter. Initially, although the absolute temperatures of the vessel are of interest, typically there is a greater interest in detecting temperature variation along the vessel. The magnitude of the temperature variations are not large and thus, the thermal sensors used in the catheter must be capable of detecting relatively small temperature variation at or about body temperature. By way of example, the literature suggests that vulnerable plaque and other tissues of interest may have temperature signatures that are on the order of 0.5 to one degree centigrade higher than surrounding tissues or less. In some situations, the temperature variations may be somewhat higher, but it is expected that in most cases, the temperature differential will be less than two to four degrees centigrade. As further research is conducted and additional indicators are identified, it is suspected that even smaller temperature differential may have diagnostic significance.

One potential treatment for vulnerable plaque is to simply stent the plaque. That is, once the vulnerable plaque has been identified with thermography, a stent delivery system can be returned to the site where the stent can then be deployed to “treat” the plaque. The act of stenting may cause the plaque to rupture but since this is a known risk, and the patient would already be on anti coagulants, it would be more of a “controlled rupture”. The patient would continue this drug treatment until risk of thrombosis due to plaque rupture was eliminated.

This can be accomplished in several ways. In one approach a thermography catheter is used to first locate the vulnerable plaque, and then a separate stent delivery catheter is used to deploy a stent at the site. In another embodiment the thermography and stenting functions are combined into one integrated device. Accordingly, in one aspect of the present invention, combined thermography and stent delivery catheters are proposed. It will become apparent to those skilled in the art that this integration can be done any number of ways utilizing common catheter design and construction techniques.

By way of example, the integrated device could consist simply of a thermography balloon (as for example described in application Ser. No. 09/346,072) with a stent crimped on the balloon. However, such a design has the drawback of the crimped stent potentially interfering with the thermal sensing. In another embodiment, the integrated device includes a “tandem” balloon catheter. That is, a catheter with a proximal balloon and a distal balloon. In this embodiment, either the proximal or the distal balloon could be the thermography balloon or the stent delivery balloon depending on the specific needs of the catheter. Once the vulnerable plaque has been identified with the proximal or distal thermography balloon, quantitative coronary angiography or QCA would be used to isolate this site, so that the proximal or distal stent delivery balloon could then be properly positioned at the site for stent deployment.

In another embodiment, the integrated thermography catheter utilizes a “rapid exchange” design. That is, the thermography catheter incorporates an auxiliary conduit that would allow a separate stent delivery catheter to be navigated to the deployment site without having to remove the thermography catheter from the guiding catheter. The proximal entrance of this auxiliary conduit is typically positioned in such a way that it is accessible with a minimal amount of repositioning of the thermography catheter.

As will be apparent to those skilled in the art, stent delivery capabilities can be combined with a wide variety of thermography devices, including any of the classes of thermography devices referenced in the background section of this application.

Another potential treatment for vulnerable plaque is to heat the region of the vulnerable plaque. The motivation and benefits of thermally heating are described, for example, in Cassells U.S. Pat. No. 5,906,636 as well as in Carl et al U.S. Pat. No. 6,047,216 and Carl et al U.S. Pat. No. 6,223,086 which are incorporated herein by reference. Accordingly, in another aspect of the present invention, combined thermography balloon and heating catheters are proposed. By way of example, combining a thermography balloon catheter with an element capable of generating infrared radiation, microwave energy, or radio frequency energy could be used to treat vulnerable plaques. That is, once the vulnerable plaque has been identified utilizing the thermography balloon embodiment of the present invention, then one of the previously mentioned heating modalities would be used to treat the vulnerable plaque.

By way of example, when utilizing infrared radiation the vulnerable plaque would be heated from 50 to 70° C. to induce apoptosis in the inflammatory cells associated with the vulnerable plaque. In an additional embodiment, an antenna generating electromagnetic energy having a frequency between 1 kHz and 30 GHz is used to heat the vulnerable plaque from between 50 to 70° C. to induce apoptosis in the inflammatory cells. Although a wide variety of electromagnetic frequencies can be used to accomplish the heating, microwave energy it typically considered to be one of the best. In addition to inducing apoptosis in the inflammatory cells, this localized heating will also cause necroses to the connective tissues in the vulnerable plaques fibrous cap, as well as soften the plaque's lipid rich core. This will in effect “stress relieve” or stabilize the plaque and fibrous cap further reducing additional risk of rupture. During this treatment phase of the procedure the thermography balloon would be used to monitor the temperature of the thermal therapies.

Additionally, a potential treatment for vulnerable plaque is to vibrationally excite the region of the vulnerable plaque using ultrasonic energy. The motivation and benefits of ultrasonic excitation are described, for example, in Brisken U.S. Pat. No. 6,210,393 and incorporated here by reference. Accordingly, in another aspect of the present invention, combined thermography balloon and ultrasonic energy catheters are proposed.

By way of example, a thermography balloon catheter with a vibrational transducer located inside the balloon would be used to first locate the vulnerable plaque, and then treat the plaque. The compression wave front of the vibrational ultrasonic energy is directed radially outward from the transducer to the previously identified vulnerable plaque so that they enter the plaque in a perpendicular fashion. This energy is used to heat the inflammatory cells from 50 to 70° C. to induce apoptosis. As previously stated, this localized heating will also cause necroses to the connective tissues in the vulnerable plaques fibrous cap, as well as soften the plaque's lipid rich core. This will in effect “stress relieve” or stabilize the plaque and fibrous cap further reducing additional risk of rupture. It will be obvious to those skilled in the art that these ultrasonic transducers can be piezoelectric, magnetostrictive or any other of a variety of commercially available transducers. Additionally, a single ultrasonic transducer or a plurality of ultrasonic transducers may be used in this embodiment of the disclosed invention.

Several presently preferred thermal mapping catheter systems and methods of thermally mapping body vessels will be described below making reference to the accompanying drawings. Generally, the described thermal mapping catheters and methods are intended to permit the diagnosis of body vessel regions that have relatively higher heat production compared with surrounding tissues and/or the temperature of adjacent luminal fluid (e.g. blood passing through an artery (vessel) being mapped). In some embodiments, thermal mapping capabilities are combined with other diagnostic or therapeutic capabilities to provide integrated tools for diagnosis and/or treatment of specific conditions. For the purpose of illustration, the inventions will be described in the context of catheters and methods suitable for thermally mapping vulnerable plaque in vascular vessels such as coronary arteries.

Generally, there are a number of considerations that must be addressed when designing a thermal mapping catheter. Initially, although the absolute temperatures of the vessel are of interest, typically there is a greater interest in detecting temperature variation along the vessel. The magnitude of the temperature variations are not large and thus, the thermal sensors used in the catheter must be capable of detecting relatively small temperature variation at or about body temperature. By way of example, the literature suggests that vulnerable plaque and other tissues of interest may have temperature signatures that are on the order of 0.5 to one degree centigrade higher than surrounding tissues or less. In some situations, the temperature variations may be somewhat higher, but it is expected that in most cases, the temperature differential will be less than two to four degrees centigrade. As further research is conducted and additional indicators are identified, it is suspected that even smaller temperature differential may have diagnostic significance.

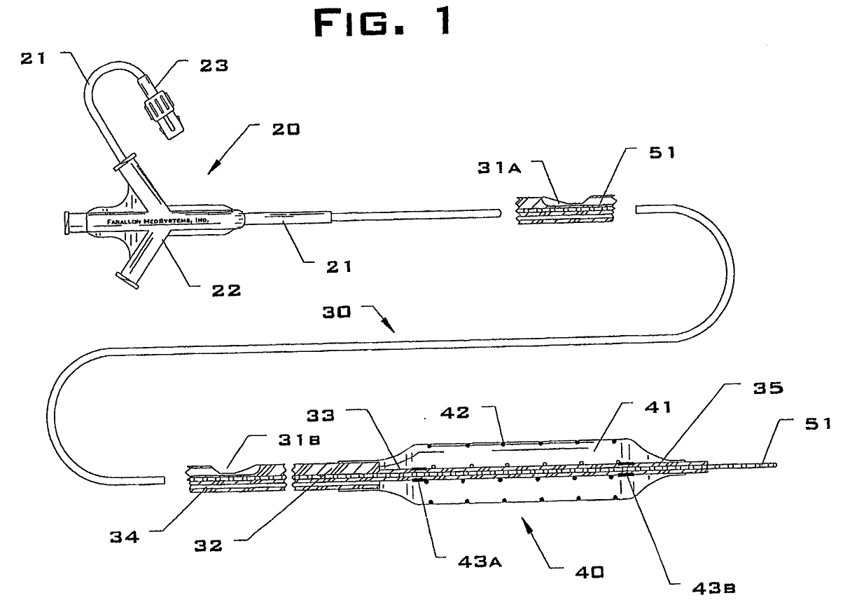

The accompanying FIGS. 1-7 illustrate various combination thermography catheters in accordance with specific embodiments of the invention. Referring initially to FIGS. 1, 3 and 4 a simple combination thermography and stent delivery catheter will be described. In this embodiment, the thermography portion of the catheter has its thermal sensors carried on an expandable balloon as described in co-pending application Ser. No. 09/346,072, which is incorporated herein by reference for all purposes. Since such thermography catheters are described in great detail in the referenced application a detailed description of their construction will not be repeated here for the sake of brevity. What is different in the present embodiment is that another lumen 35, referred to here in as a stent delivery lumen is formed in the catheter shaft. The lumen 25 has a proximal entrance port 31(a) and a distal exit port 31(b). A conventional small diameter stent delivery device can then be inserted into the stent delivery lumen through the proximal entrance port 31(a) and out the distal exit port 31(b) and deployed in a conventional manner. As will be understood by those skilled in the art, some of the existing stent delivery devices are very small in diameter and can readily be deployed in this manner.

Of course, the location of the entrance and exit ports for the stent delivery lumen can be widely varied. In the illustrated embodiment, the entrance port 31(a) is located distally of the multi-arm connector 22. In alternative embodiments, entrance to the shaft can be by way of the multi-armed connector (which would need to be modified accordingly), through a separate connector (not shown), or through a port located proximally of the multi-armed connector. Similarly, the location of the exit port 31(b) can be widely varied as well. By way of example, it may be located proximally, distally or intermediate relative to the thermal sensors 42. Ports located distally of the thermal sensors can open either to the side of the catheter as the illustrated port 31(b) does, or open distally at the distal tip of the catheter. In embodiments that utilize a guide wire, after the catheter is positioned, the guide wire could be withdrawn and the stent delivery catheter inserted in its place. That is, the guide wire lumen can double as the stent delivery lumen.

It should be apparent that the described stent delivery lumen can be incorporated into virtually any type of thermography catheter, including any of the designs described in the background section of this application. This can typically be done making only relatively minor changes to the design of the catheters.

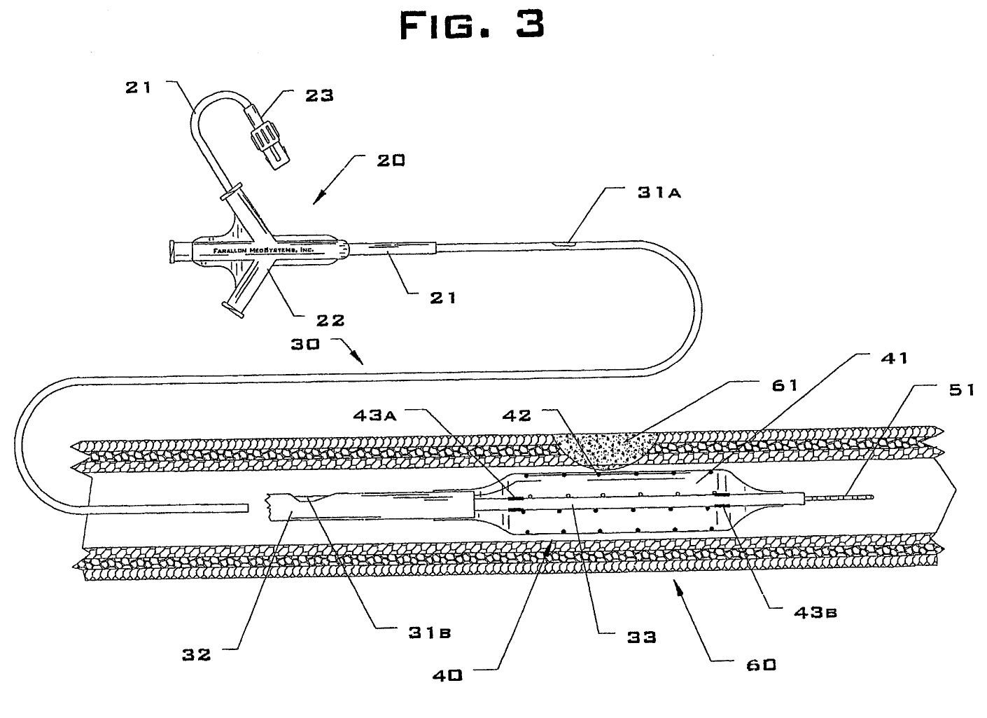

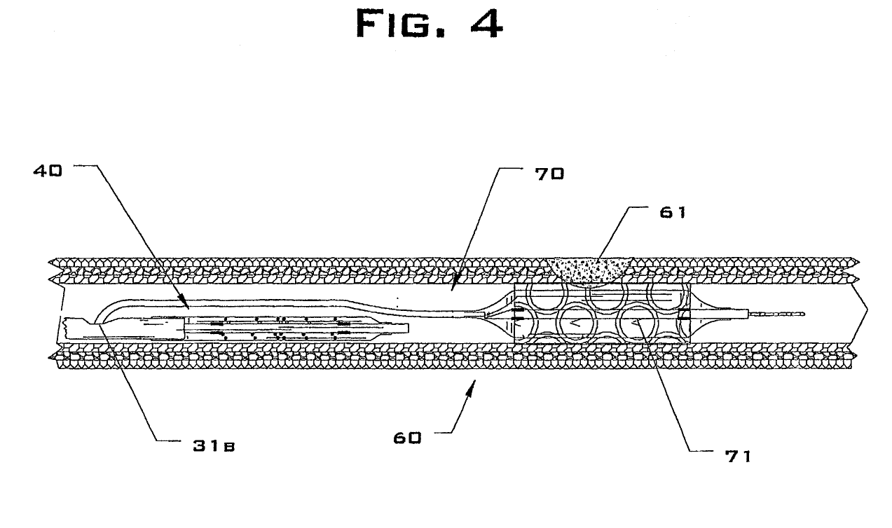

In operation, the thermal sensors (e.g. sensors 42) are used to locate vulnerable plaque as illustrated in FIG. 3. The thermography catheter can then be pulled back and the stent deliver device 70 inserted through the stent delivery lumen 35 and out the exit port 31(b). The stent 71 carried by the stent delivery device 70 is then positioned at the location of the identified vulnerable plaque (or other region that is desired to be stented) and the stent 71 is deployed in a conventional manner. The deployment of the stent 71 in the region of the vulnerable plaque is illustrated in FIG. 4.

It should be appreciated that stenting vulnerable plaque has the potential to cause rupture of the plaque. Thus, in many cases it will be desirable to administer appropriate anti-thrombogenic (anti-clotting) agents. Such agents can be delivered either locally by the catheter (as for example, by fluid delivery mechanisms such as those described in the referenced application) or systemically.

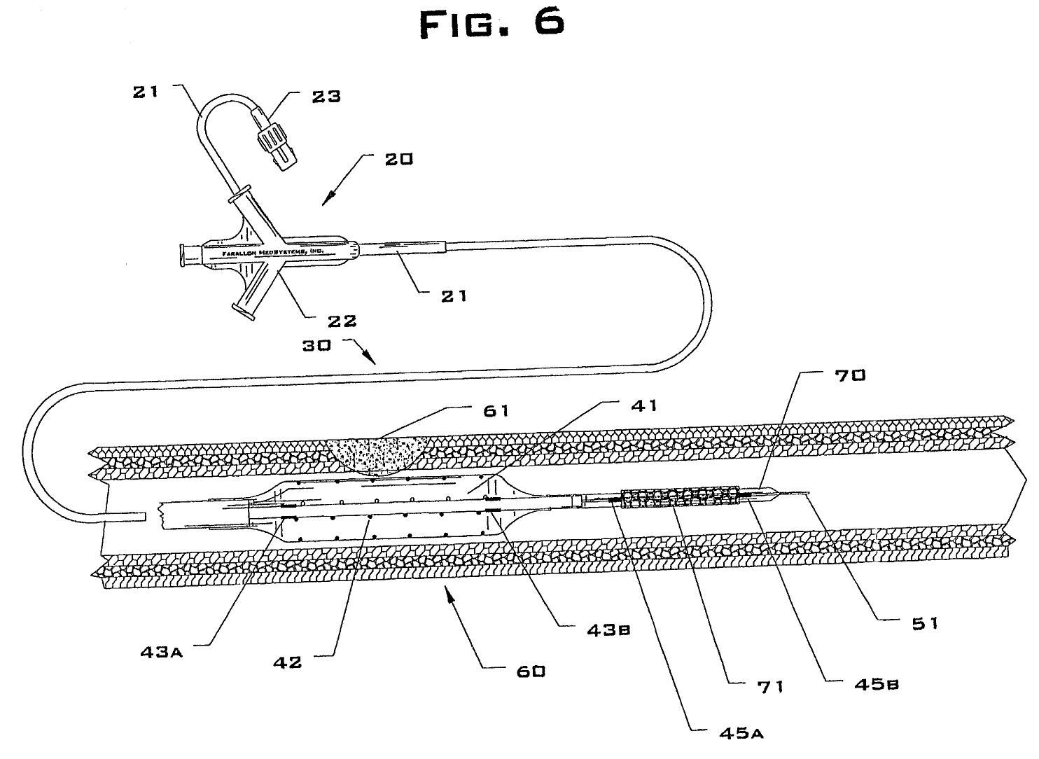

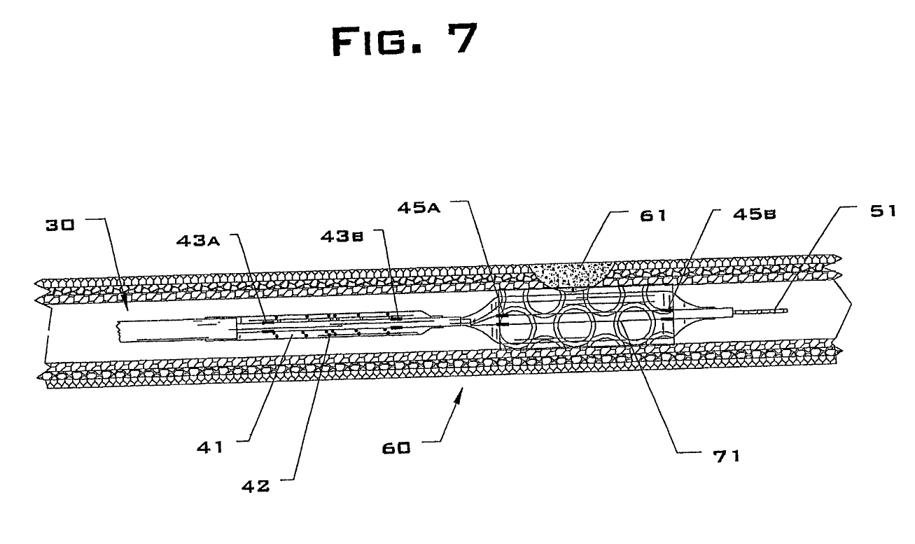

Another embodiment of a combined thermal mapping and stent delivery catheter is illustrated in FIGS. 6 and 7. In this embodiment, the stent delivery mechanism (with appropriate marker bands) is integrally formed or carried on the catheter itself. In the embodiment shown, the stent delivery mechanism is located distally relative to the thermal sensing balloon 41. Of course in alternative embodiments, the stent delivery mechanism could be located proximal relative to balloon 41. In this embodiment, when vulnerable plaque is identified, the thermography catheter is pulled back an appropriate amount and a stent delivery balloon is inflated (or other suitable deployment device actuated) to deploy the stent 71 as illustrated in FIG. 7.

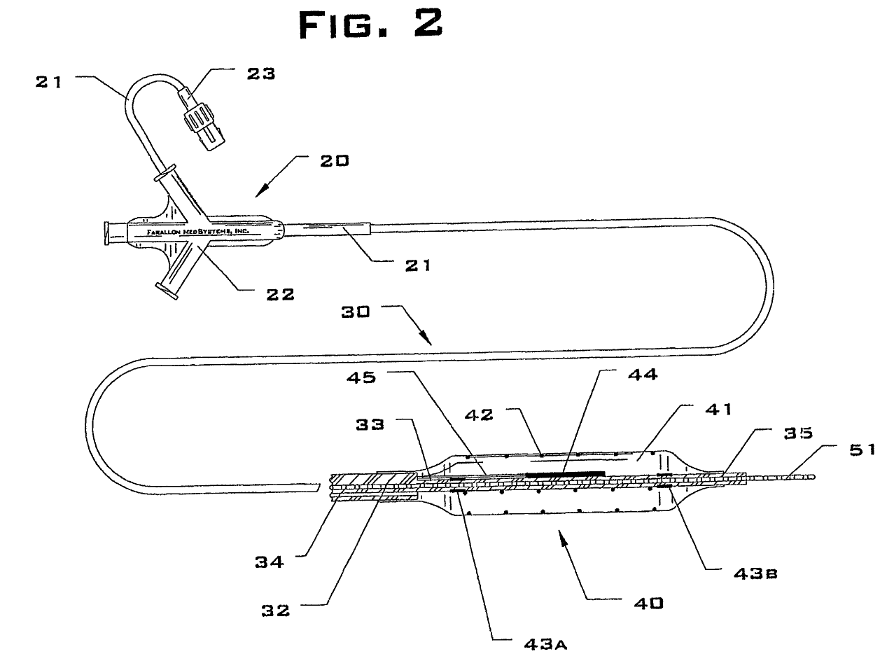

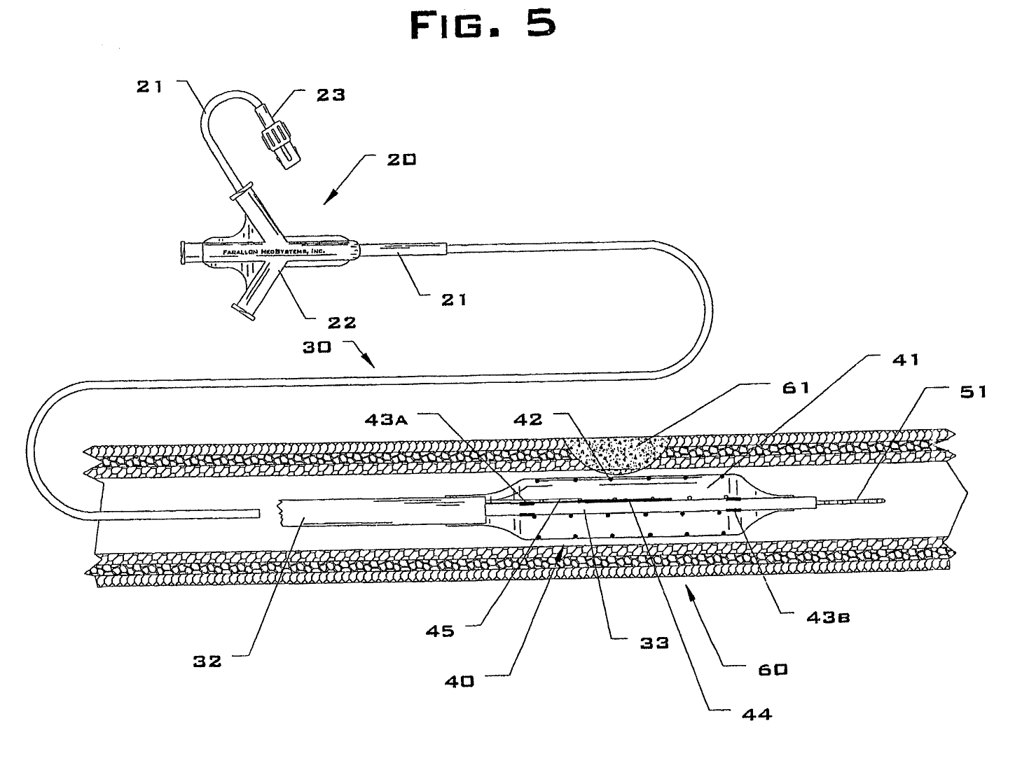

A second treatment approach is to thermally heat the walls of the artery. It has been suggested that thermally heating the walls of an artery may have an advantageous therapeutic effect. A representative catheter design that combines thermography and thermal heating capabilities is illustrated in FIG. 2 and FIG. 5. In this embodiment, the heating element 44, may take the form of a passive resistor used to heat the fluid within the thermal sensor carrying balloon 41 used to position the thermal sensors 42. Of course, the thermal sensors 42 can be used to monitor the temperature of the balloon 41 and/or adjacent vessel walls. When vulnerable plaque or other regions to be treated with heat are identified, current can be delivered to the resister wires 45 through conductive wires that pass through the catheter shaft 30. The resistor then heats the fluid within the inflated balloon 41, which heats the adjacent vessel walls.

In a second embodiment the heating element 44 shown in FIG. 2 and FIG. 5 may take the form of an infrared emitting element, to heat the fluid within the sensor carrying balloon 41. In a third embodiment the heating element 44 shown in FIG. 2 and FIG. 5 may take the form of a microwave or radio frequency emitting antenna. Additionally, in a fourth embodiment the heating element 44 shown in FIG. 2 and FIG. 5 may take the form of an ultrasonic transducer.

The actual temperature that the vessel walls are heated to will depend in large part on the desired effect. In one application, the vessel walls will be heated to a temperature of between about 50 and 70 degrees centigrade with the temperature and duration being selected so that inflammatory cells within the muscle walls are killed or sufficiently damaged, without killing or otherwise permanently damaging the smooth muscle cells in the artery walls.

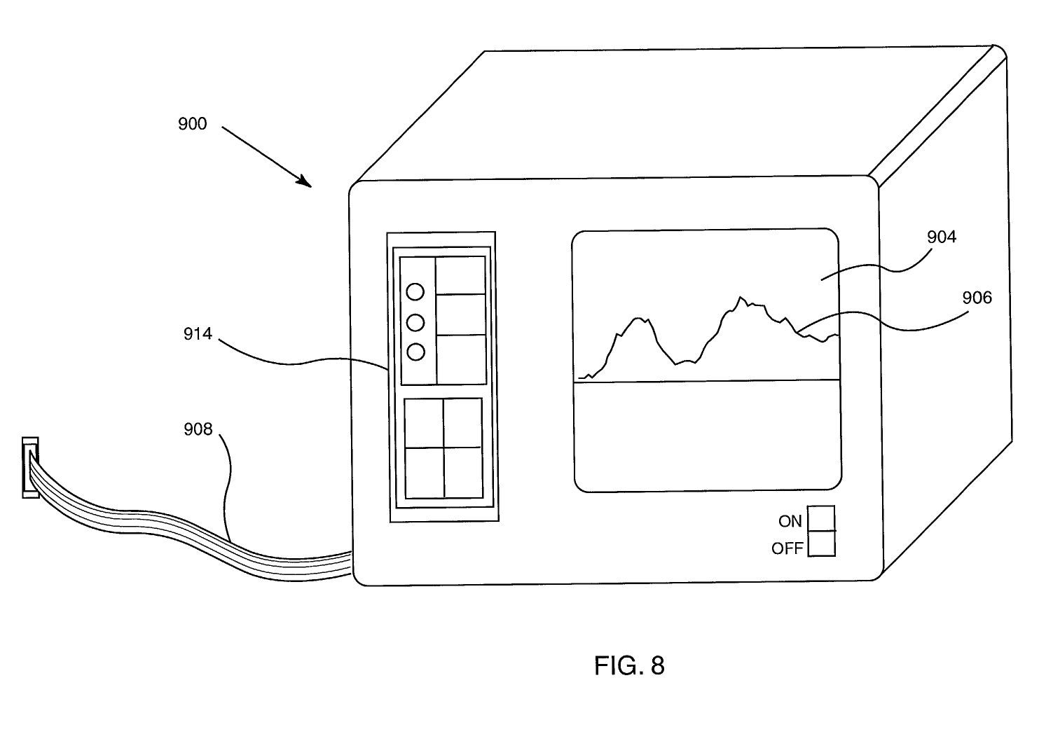

Referring next to FIG. 8, a monitor suitable for displaying thermal maps will be described. The monitor 900 includes a display screen 904 suitable for displaying a thermal map 906. The monitor also includes a connector 908 that couples to the electrical connector 818 on the hub assembly and a number of control buttons 914. Suitable hub arrangements are provided at the proximal end of the catheters. As will be appreciated by those skilled in the art, the construction of the proximal hub assemblies can and will vary widely depending on the needs of a particular system. Generally, the hub must include appropriate electrical connectors for the thermal sensors and fluid connectors for the fluid delivery tubes. In embodiments that are designed to pass a stent delivery device or a guide wire, it also includes a valve (such as a Tuohy Borst valve) suitable for passing the stent delivery device or guide wire and providing a fluid seal around the guide wire.

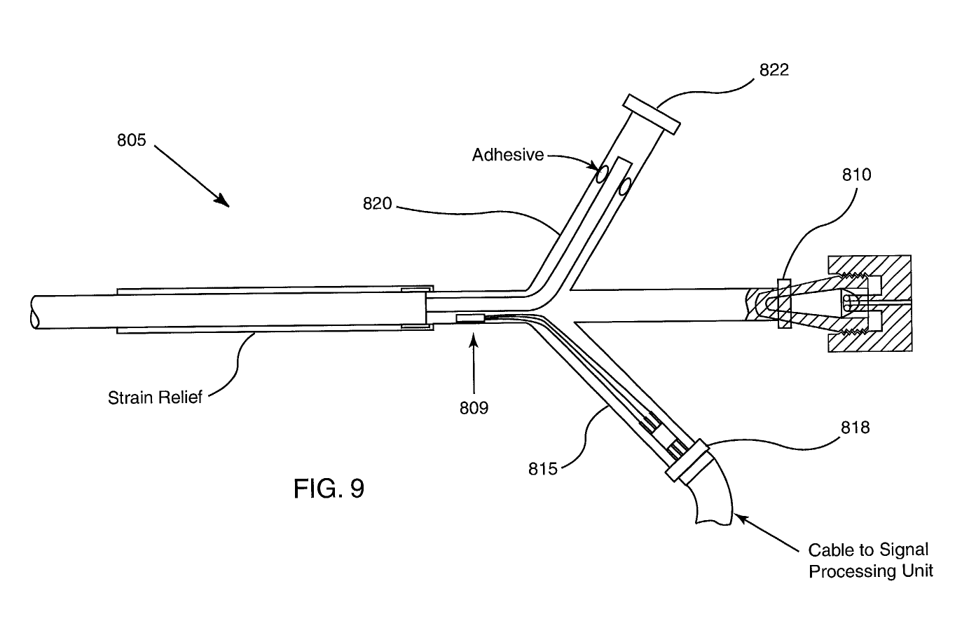

FIG. 9 illustrates one representative hub assembly that may be used in conjunction with some of the described catheters. In the embodiment shown, the proximal hub 805 includes a central arm 809 having a guide wire and/or stent delivery device valve 810, an electrical sensor arm 815 having an electrical connector 818, and an inflation arm 820 having a luer connector 822. The central arm extends straight from the catheter to facilitate insertion of the guide wire therethrough. Conventional guide wire valves such as a Tuohy Borst valve can be used to create a fluid seal. The electrical connector 818 couples the thermal sensor wires to an appropriate interconnect cable attached to the data acquisition instrumentation (which preferably includes a display as illustrated in FIG. 8). By way of example, a conventional Lemo® multi-pin connector works well as the electrical connector 818. The luer connector 822 provides a fluid seal between the inflation device and the balloon inflation lumen of the catheter.

Of course, in embodiments that include infusion and/or withdrawal capabilities, additional arms would need to be provided to facilitate appropriate fluid communication pathways between the catheter and external controller and/or pumps.

Similarly, if separate inflation and deflation conduits are provided, it may be desirable to provide additional hub arms to facilitate these connections as well.

Although only a few embodiments of the present invention have been described in detail, it should be understood that the present invention may be embodied in many other specific forms without departing from the spirit or scope of the invention. The described interventional tools can be provided or combined with a number of other capabilities beyond the stenting and thermal heating capabilities described in some detail above. By way of example, imaging capabilities, such as ultrasonic imaging, angioscopy or angiography may be desirable. In other applications, it may be desirable to combine the thermal mapping with the delivery and/or withdrawal of various fluids (such as therapeutic agents). Suitable structures for some such devices are described in detail in application Ser. No. 09/346,072 which are incorporated herein by reference for all purposes.

As will be appreciated by those skilled in the art, the literature suggests that vulnerable plaque and other tissues of interest may have temperature signatures that are on the order of 0.5 to one degree centigrade higher than surrounding tissues or less. In some situations, the temperature variations may be somewhat higher, but it is expected that in most cases, the temperature differential will be less than two to four degrees centigrade. As further research is conducted and additional indicators are identified, it is suspected that even smaller temperature differential may have diagnostic significance. In the embodiments shown, the thermal sensors are generally arranged in uniformly spaced rows and/or bands and typically carried by an inflatable balloon. However, it should be apparent that the sensors could be arranged in a wide variety of patterns, including both non-uniformly spaced and non-aligned patterns without departing from the spirit of the invention. In some embodiments, it may be preferable to provide a single or a small number of thermal sensors (such as a band of sensors) that are then “dragged” or “pushed” through the vessel to facilitate thermal mapping. Although the described inflatable balloon for placing the thermal sensors into engagement with or proximity to the vessel walls works well, in other embodiments, the thermal sensor(s) may be placed in a variety of other locations. These alternative placements may include on the catheter itself, or on a different type of expandable or extendable device. Further, although specific thermal mapping catheter constructions have been described, components of the various designs may in many cases be mixed and matched as appropriate to meet the needs of a particular application.

The examples above utilize thermisters or thermocouples as the thermal sensors. It should be appreciated that a variety of sensors may be used alternatively, including infrared sensors, luminescence absorption sensors and thermal cameras. However, thermisters and thermocouple-based systems are particularly advantageous because of their compactness and simplicity of function. Thermisters in particular have a reputation for very high sensitivity and are available in very small sizes. Thermocouples are somewhat less sensitive than thermisters, but are known for durability and very small size.

Virtually any type of stent may be delivered by the described stent delivery devices. These may include stents that are coated with therapeutic agents, diagnostic, marking agents, radioactive agents or any other type of agent that may be appropriate for a particular application. From the forgoing, it should be apparent that the present examples are to be considered as illustrative and not restrictive, and the invention is not to be limited to the details given herein, but may be modified within the scope of the appended claims.

1. An interventional tool suitable for measuring the temperature of or temperature variations in a vessel wall in the body of a patient, the interventional tool comprising:
an elongated member suitable for insertion in a vessel in the body of a patient, the elongated member having proximal and distal ends;
a thermal sensor carried by the elongated member, the thermal sensor being suitable for detecting the temperature of walls of a vessel the elongated member is inserted into to facilitate the detection of vulnerable plaque within the vessel; and
a stent delivery device carried by the elongated member, the stent delivery device being suitable for delivering a stent to a selected segment of a vessel the elongated member is inserted into.

2. An interventional tool as recited in claim 1 wherein the stent delivery device includes an expander for expanding a stent carried by the elongated member.

3. An interventional tool as recited in claim 2 further comprising an expandable stent positioned to be delivered by the stent delivery device.

4. An interventional tool as recited in claim 1 wherein the interventional tool takes the form of a catheter and the elongated member is a flexible tubular member, the interventional tool further comprising an expansion device carried by the elongated member that carries the thermal sensor, the expansion device being suitable for positioning the thermal sensor adjacent the vessel wall.

5. An interventional tool as recited in claim 4 wherein the expander includes a first balloon.

6. An interventional tool as recited in claim 4 wherein the expander includes a first balloon and a sheath member formed from a second balloon material; and
a plurality of additional thermal sensors are provided, the thermal sensors being sandwiched between the first and second balloons.

7. An interventional tool comprising:
an elongated member suitable for insertion in a vessel in the body of a patient, the elongated member having proximal and distal ends;
at least one thermal sensor carried by the elongated member, each thermal sensor being suitable for detecting the temperature of walls of a vessel the elongated member is inserted into to facilitate the detection of vulnerable plaque within the vessel; and
a deployment lumen within the elongated member, the deployment lumen being sized suitably for receiving a stent delivery catheter therethrough, the deployment lumen having a distal port that permits the distal portion of the stent delivery catheter to pass therethrough to exit the elongated member to permit deployment of a stent.

8. An interventional tool as recited in claim 7 further comprising at least one infusion port suitable for delivering therapeutic or diagnostic agents into a vessel.

9. An interventional tool as recited in claim 8 wherein at least some of the infusion ports are located between adjacent thermal sensors.

10 A thermal mapping system including:
a interventional tool as recited in claim 1; and
a display device arranged to receive the signals from the thermal sensors and display a thermal map of a longitudinal section of the vessel that shows temperature variations along the vessel to facilitate identifying a region of vulnerable plaque.

11. A method of thermally mapping and treating a vessel comprising:
inserting a catheter having thermal sensors into the vessel;
identifying a region of vulnerable plaque by sensing temperatures or temperature variations along the using the thermal sensors; and
delivering a stent carried by or inserted through the catheter to the identified region of vulnerable plaque.

12. A method of thermally mapping and treating a vessel comprising:
inserting a catheter having thermal sensors into the vessel;
identifying a region of vulnerable plaque by sensing temperatures or temperature variations along the using the thermal sensors; and
using the catheter to heat the identified region of vulnerable plaque to induce apoptosis in inflammatory cells associated with the vulnerable plaque.

13. An interventional tool suitable for measuring the temperature of or temperature variations in a vessel wall in the body of a patient, the interventional tool comprising:
an elongated member suitable for insertion in a vessel in the body of a patient, the elongated member having proximal and distal ends;
a thermal sensor carried by the elongated member, the thermal sensor being suitable for detecting an indication of the temperature of or temperature variations in walls of a vessel the elongated member is inserted into to facilitate the detection of vulnerable plaque within the vessel;
an expansion device carried by the elongated member, the expansion device being suitable for positioning the thermal sensor against the vessel wall; and
a heater arranged to heat a selected vulnerable plaque containing segment of a vessel the elongated member is inserted into to a temperature sufficient to induce apoptosis in inflammatory cells associated with the vulnerable plaque.

14. An interventional tool as recited in claim 13 wherein the heater is a heating means for delivering heat to the selected segment of the vessel. 15. An interventional tool as recited in claim 13 wherein the heater is an antenna suitable for delivering electromagnetic energy to facilitate heating.

16. An interventional tool as recited in claim 13 wherein the heater is a resistive heater.
